(19) United States
(12) Patent Application Publication
(10) Pub. No.: US 2022/0095993 A1
(43) Pub. Date: Mar. 31, 2022

(54) METHODS AND SYSTEMS FOR FORECASTING EPILEPTIC EVENTS

(71) Applicant: Seer Medical Pty Ltd, Melbourne (AU)

(72) Inventors: Philippa Karoly, Melbourne (AU); Dean Freestone, Melbourne (AU); Mark Cook, Melbourne (AU)

(21) Appl. No.: 17/484,979

(22) Filed: Sep. 24, 2021

(30) Foreign Application Priority Data
Sep. 25, 2020 (AU) 2020903470

(51) Int. Cl. A61B 5/00; A61B 5/372; A61B 5/0205; G16H 50/70; G16H 50/20; G16H 20/13

(52) U.S. Cl. CPC A61B 5/4094; A61B 5/372; A61B 5/7275; A61B 5/7278; A61B 5/746; A61B 5/02405; A61B 5/0205; G16H 50/70; G16H 50/20; G16H 20/13; A61B 5/725

(57) ABSTRACT

A method comprises determining historical data associated with a subject experiencing epileptic events over a first time period, the historical data comprising non-EEG physiological data recorded over the first time period, and a time at which epileptic events occurred during the first time period. The method further comprises extracting from the non-EEG physiological data, one or more temporal models indicative of a subject specific cycle; and generating one or more temporal probabilistic models based on the respective one or more temporal models, the non-EEG physiological data, and the times at which each epileptic event occurred, wherein each temporal probabilistic model is representative of a probability of future seizure activity in each of a plurality of time windows. The method further comprises providing the one or more temporal probabilistic models for determining an estimate of seizure probability in the subject for one or more of the plurality of time windows.

### TECHNICAL FIELD

The present disclosure relates to methods and systems for generating models for forecasting epileptic events, such as seizures, and in some embodiments, for forecasting seizure likelihood in subjects with epilepsy using probabilistic modelling.

### BACKGROUND

Cyclic phenomena are ubiquitous across biological systems. In the field of chronobiology, circadian (and related-24 hour rhythms) have been widely studied; however, other time scales, including weekly (circaseptan), monthly (circalunar or circatringian), seasonal (circannual) and even longer rhythms have also been implicated across a diverse range of physiological functions. Recent technological developments have rapidly advanced the capacity to measure human rhythms over very long time scales. In neurology, huge strides have been made in chronic brain recording devices, leading to overwhelming evidence that multiday cycles govern excitability in the epileptic brain. The phenomena of multiday cycles in epilepsy was identified centuries earlier through observation of individual's seizure patterns [“Griffiths”, Griffiths G, Fox J T. Rhythm in epilepsy. The Lancet 1938; 232: 409-16]. Subsequent studies using chronic electroencephalography (EEG) have shown that the periodicity of seizure occurrence is underpinned by patient-specific circadian and multiday (5-30 day) rhythms of epileptic activity [“Baud”, Baud M O, Kleen J K, Mirro E A, et al. Multi-day rhythms modulate seizure risk in epilepsy. Nat Commun 2018; 9: 88, “Karolyl”, Karoly P J, Freestone D R, Boston R, et al. Interictal spikes and epileptic seizures: their relationship and underlying rhythmicity. Brain 2016; 139: 1066-78] and cortical excitability [“Maturana”, Maturana M I, Meisel C, Dell K, et al. Critical slowing down as a biomarker for seizure susceptibility. Nat Commun 2020; 11: 2172]. Importantly, multiday cycles in epilepsy exist for most people [Baud, “Karoly2”, Karoly P J, Goldenholz D M, Freestone D R, et al. Circadian and circaseptan rhythms in human epilepsy: a retrospective cohort study. The Lancet Neurology 2018; 17: 977-85, Maturana], and appear to be ‘free-running’ in the sense that they are not tied to environmental cues (weekday, lunar cycle, calendar), equally prevalent for men and women [Griffiths, Baud, Karoly2], and across epilepsy syndromes and seizure types [Karoly2]. The content of each of Griffiths, Baud, Karoly1 and Karoly2 is incorporated herein by reference.

The periods of highest seizure likelihood vary greatly between patients, but on an individual level remain consistent over many years. As described in International (PCT) Patent Application Number PCT/AU2018/050575, the entire content of which is incorporated herein by reference, a temporal model representing a probability of a future seizure occurrence can be determined from historical data associated with epileptic events experienced by the subject over a period of time, and specifically the time at which each epileptic event over the time period.

The unpredictability of seizures has a profound impact on their safety of people with epilepsy. The temporal model described in PCT/AU2018/050575 provides accurate seizure forecasting with the potential to greatly improve an individuals' quality of life, to enable pre-emptive administration of therapies and/or allow steps to ensure personal safety to be undertaken.

Nonetheless, it is desirable to provide an improved, or at least an alternative method for forecasting seizures in subjects.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

According to an aspect of the disclosure, there is provided a method comprising determining historical data associated with a subject experiencing epileptic events over a first time period, the historical data comprising non-EEG physiological data recorded over the first time period, and a time at which epileptic events occurred during the first time period; extracting from the non-EEG physiological data, one or more temporal models indicative of a subject specific cycle; generating one or more temporal probabilistic models based on the respective one or more temporal models, the non-EEG physiological data, and the times at which each epileptic event occurred, wherein each temporal probabilistic model is representative of a probability of future seizure activity in each of a plurality of time windows; and providing the one or more temporal probabilistic models for determining an estimate of seizure probability in the subject for one or more of the plurality of time windows.

In some embodiments, the method further comprises determining the estimate of seizure probability in the subject for one or more of the plurality of time windows using the one or more temporal probabilistic models. In some embodiments, the method further comprises outputting an alert based on the estimate of seizure probability in the subject for one or more of the plurality of time windows.

The alert may comprise one or more of: (i) the estimate of seizure probability in the subject for one or more of the plurality of time windows; (ii) a seizure occurrence risk rating; (iii) information concerning a cause of risk elevation; (iv) a recommendation to take or modify change medication or therapy; and (v) a recommendation to alter one or more parameters of a therapeutic device for delivering stimulation to the subject. The method may further comprise scheduling administration of medication based on the estimate of seizure probability in the subject.

In some embodiments, generating the one or more temporal probabilistic models comprises: filtering the non-EEG physiological data into one or more component frequencies corresponding to the one or more temporal models to produce one or more respective filtered non-EEG physiological data; determining a phase of each of the filtered non-EEG physiological data; and mapping the times of the epileptic events to the phase of each of the filtered non-EEG physiological data.

The non-EEG physiological data may comprises cardiac output recorded over the first time period. The cardiac output may comprise one or more of: (i) heart rate, and (ii) heart rate variability. The non-EEG physiological data may comprise values of one or more variables of sleep recorded over the first time period. The one or more sleep variables may comprise one or more of historical times of first waking and sleeping, time of hours awake over a previous time period, hours asleep over a previous time period, and sleep depth. The non-EEG physiological data may comprise values of one or more variables of activity recorded over the first time period. The non-EEG physiological data comprises one or more of: (i) values of one or more variables of oxygen saturation recorded over the first time period; (ii) values of one or more variables of electrodermal activity recorded over the first time period; (iii) values of one or more variables of skin temperature recorded over the first time period; and (iv) values of one or more variables of respiratory rate recorded over the first time period.

The non-EEG physiological data may comprise an electrocardiograph (ECG) received from the heart of the subject. The non-EEG physiological data may comprise a photo-plethysmograph signal received from the heart of the subject. The non-EEG physiological data may comprise an actigraphy received from the subject.

The method may further comprise determining updated historical data associated with the subject experiencing epileptic events over a second time period, the updated historical data comprising updated non-EEG physiological data recorded over a second time period and a time at which epileptic events occurred during the second time period; extracting from the updated non-EEG physiological data, one or more updated temporal models indicative of the subject specific cycle; generating one or more updated temporal probabilistic models based on the respective one or more updated temporal models, the updated non-EEG physiological data, and the times at which each epileptic event occurred during the second period of time, wherein each updated temporal probabilistic model is representative of a probability of future seizure activity in each of a plurality of time windows; and providing the one or more updated temporal probabilistic models for determining an estimate of seizure probability in the subject for one or more of the plurality of time windows. The method may further comprise receiving new seizure data for the second period of time and responsive to receiving the new seizure data, determining the updated historical data. The second time period may include the first time period.

According to another aspect of the disclosure, there is provided an seizure forecasting system comprising one or more processors, and memory comprising computer executable instructions, which when executed by the one or more processors, causes the system to perform any one of the described methods.

In some embodiments, the seizure forecasting system may comprise one or more measurement devices configured to record the non-EEG physiological data. The one or more measurement devices comprises one or more of the following for recording the physiological data: a heart rate monitor; a blood pressure monitor; a sweat sensor; an accelerometer; and a gyroscope.

According to another aspect of the disclosure, there is provided a non-transitory computer readable storage medium seizure comprising instructions, which when executed by one or more processors, are configured to perform any one of the described methods.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure will now be described by way of non-limiting example only with reference to the accompanying drawings, in which:

FIG. 1 is a schematic diagram illustrating methods for generating and evaluating temporal model(s) for estimating a probability of epileptic event occurrence in a subject having epilepsy, according to some embodiments;

FIG. 2 is a schematic illustration of a seizure forecasting system according to some embodiments of the disclosure;

FIG. 3 is a schematic illustration of a device configured to generate alerts based on one or more subject specific temporal probabilistic models;

FIG. 4 is a process flow diagram of a method for generating one or more temporal probabilistic models for forecasting future seizure activity in a subject having epilepsy, according to some embodiments;

FIGS. 5A and 5D are graphical illustrations of multiday heart rate cycles over time for two individuals of the study as measured from photo-plethysmography (PPG);

FIGS. 5B and 5E depict subject specific temporal models for the two individuals, respectively, as extracted from the plots of FIGS. 5a and 5d;

FIGS. 5C and 5F depict wavelet power spectrum of the heart rate plots of FIGS. 5A and 5B;

FIGS. 6A and 6B are plots of a distribution of heart rate cycles over time for two individuals, averaged over the cohort of the study;

FIGS. 6C and 6D show the prevalence of multiday heart rate cycles across the cohort of the study;

FIG. 7 graphically illustrates five examples of seizure occurrence locked to heart rate cycles;

FIGS. 8A to 8D graphically illustrates synchronisation index (SI) values across the cohort for different cycles; and

FIG. 9 illustrates three plots of heart rate measured from ECG versus time for three example cases where different weekly trends were observed;

FIG. 10 graphically illustrates epileptic activity locked to circadian cycles of heart rate, HRV and time of day; and

FIG. 11 is a schematic illustration of a system for generating temporal probabilistic model(s) for forecasting future seizure activity in a subject having epilepsy, according to some embodiments.

### DETAILED DESCRIPTION

Embodiments of the present disclosure provide a probabilistic approach to forecasting seizure likelihood in subjects with epilepsy that incorporates prior knowledge about underlying patterns in seizure occurrence with respect to other variables that affect the probability of a subject or patient having a seizure.

Multiday cycles of cortical excitability have been predominantly investigated in people with epilepsy. As set out in the described embodiments, it has been established that, analogous to circadian rhythms, free-running multiday rhythms are widespread across the major organ systems and that non-electroencephalography (EEG) physiological data (i.e., physiological data that is not derived from a brain of the subject) as well as EEG physiological data correlates with seizure occurrence. For example, suitable non-EEG physiological data may include cardiac data, such as heart rate and/or heart rate variation, sleep data, oxygen saturation data, electrodermal activity data, respiratory data, and/or activity data. In one study, as described below, it has been established that cardiac output shows multiday rhythms akin to cycles of cortical excitability. Furthermore, the inventor recognises that there can be differing seizure risk factors (for example, increased heart rate, decreased heart rate variability, sleep deprivation etc.) between people with epilepsy.

The present disclosure relates to methods and system for generating one or more temporal probabilistic models, each representative of a probability of future epileptic event occurrence. The temporal probabilistic model(s) are based on respective temporal models indicative of specific cycles of a subject extracted from the non-EEG physiological data of the subject. The temporal probabilistic model(s) estimate seizure occurrence probability in the subject and allow for accurate seizure occurrence forecasting for the subject. This provides a groundbreaking new avenue to monitor aspects of epilepsy without the need for neural implants.

### Overview

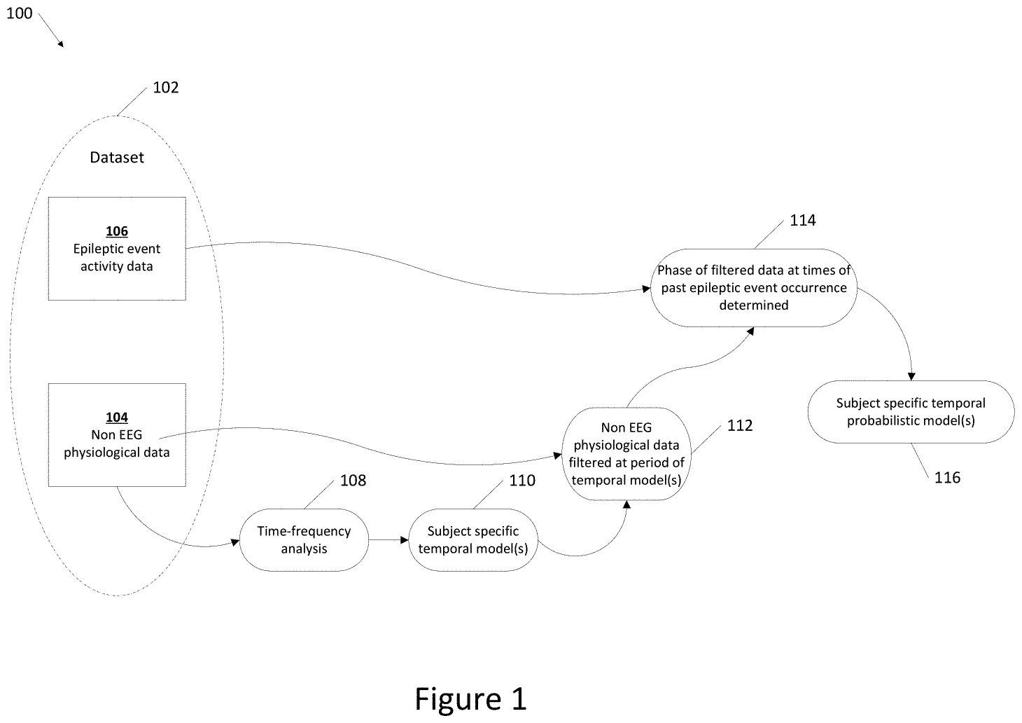

Referring to FIG. 1, there is shown a schematic 100 illustrating methods for generating and evaluating temporal model(s) for forecasting future seizure activity in a subject having epilepsy, according to some embodiments.

The schematic 100 illustrates a dataset of historical data 102 associated with a subject with epilepsy. The historical data 102 is recorded over a period of time and comprises non-EEG physiological data 104, and seizure activity data 106. The seizure activity data 106 comprises a time (that is a time of day and date) at which each seizure or seizures occurred during the time period.

In some embodiments, the seizure activity data 106 is determined or captured in any suitable manner. For example, the seizure activity data 106 may be determine from data recorded by the patient, for example, keeping a seizure occurrence diary and/or from clinical records. The seizure activity data 106 may be derived from physiological signals, which may be performed automatically, and/or may be detected from electrodes or an implantable device. For example, seizure activity data 106 may be determined or captured by analysing EEG data indicative of brain activity of the patient.

A time-frequency analysis 108 is performed on the non-EEG physiological data 104 to detect subject specific temporal model(s) 108 associated with the subject, that is, cycles at a given time period. For example, the time-frequency analysis 108 may involve detecting periods (circadian, ultradian, and/or infradian) with significant peaks, each significant peak corresponding to a temporal model 108. The non-EEG data signal is then filtered at the period of the temporal model(s) 110 to produce filtered non-EEG physiological data signal(s) 112.

A phase 114 of the non-EEG physiological data signal(s) 112 at times of past epileptic event occurrences, derived from the seizure activity data 106, is then determined and used to generate the temporal probabilistic model(s) 116.

### Seizure Forecasting System

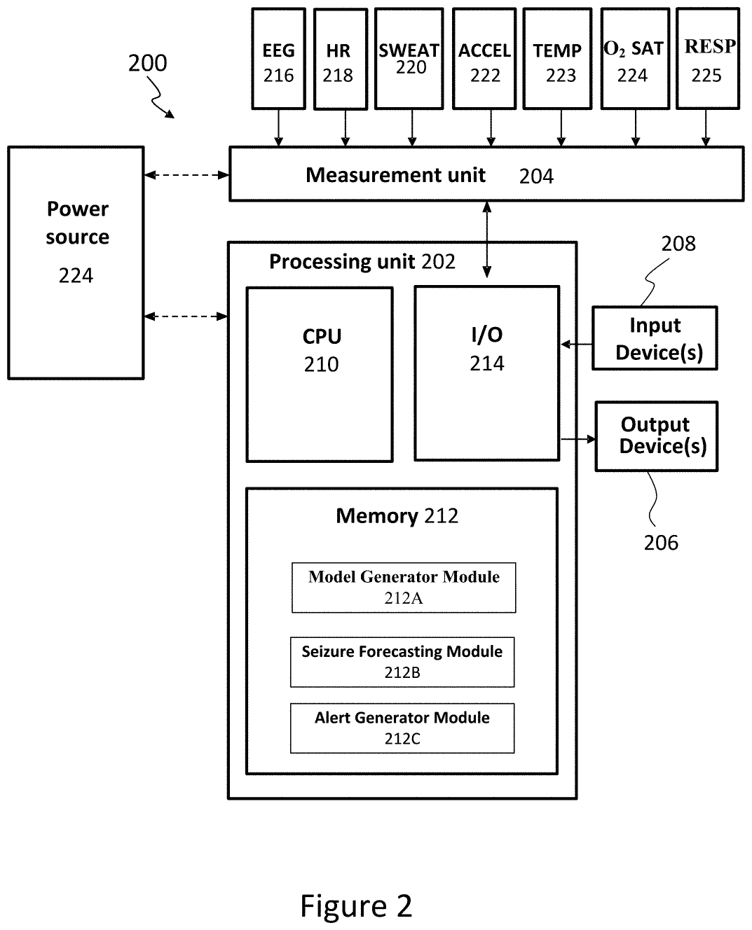

An example seizure forecasting system 200 according to an embodiment of the disclosure is illustrated in FIG. 2. The system 200 comprises a processing unit 202 comprising a central processing unit (CPU) 210, memory 212, and an input/output (I/O) bus 214 communicatively coupled with one or more of the CPU 210 and memory 212.

Memory 212 may be configured to store physiological data and seizure activity data 106 associated with a particular subject. The seizure activity data 106 may be derived from physiological signals such as EEG signals, from patient or clinical records, or may be may be detected from electrodes or an implantable device.

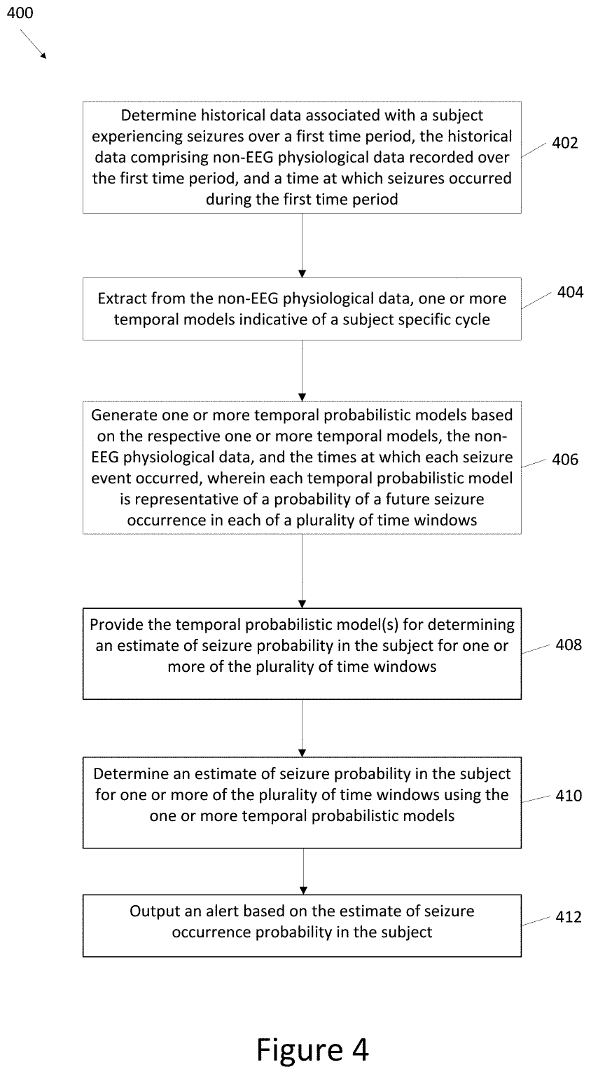

Memory 212 may further comprise computer executable instructions (for example, a temporal probabilistic model generator module 212A), which when executed by the CPU 210, cause the processing unit 202 to perform a method for generating one or more temporal probabilistic models for determining a probability of a future seizure occurrence in each of a plurality of time windows, as described in more detail below with reference to FIG. 4. The method 400 may be performed iteratively. For example, the processing unit 202 may be performed to generate one or more updated temporal probabilistic models for a particular subject in response to receipt by the processing unit 202 of new seizure activity data 106 associated with the particular subject.

The system 200 may further comprise a measurement unit 204. The measurement unit 204 may be coupled to one or more devices for recording non-EEG physiological data, and in some embodiments, also EEG physiological data. Measurement devices which may be coupled to the measurement unit 204 may comprise (but are not limited to) an EEG monitoring device 216, a heart monitor (photo-plethysmograph or ECG) 218, a sweat or electro dermal sensor 220 an accelerometer 222 (or similar motion detector), a temperature sensor 223, and oxygen saturation measurement device 224, such as a pulse oximeter, and a respiratory monitor 225. Other examples of measurement devices which may be coupled to the measurement unit 204 include blood pressure monitors, glucose monitors, cortisol sensor, and gyroscopes etc.

The measurement unit 204 may comprise one or more amplifiers and/or digital signal processing circuitry for processing signals received from the one or more measurement devices 216, 218, 220, 222, 223, 224, 225. Such signal processing circuitry may include, for example, sampling circuits for sampling signals received from the one or more measurement devices 216, 218, 220, 222, 223, 224, 225 as well as filters for filtering such signals in accordance with embodiments described above. The measurement unit may also be configured to extract and process information received from the one or more measurement devices 216, 218, 220, 222, 223, 224, 225. To that end, the measurement unit 204 may include memory to store data received from the one or more measurement devices 216, 218, 220, 222, 223, 224, 225.

The EEG monitoring device 216 may comprise one or more electrode leads each comprising one or more electrodes. Such leads may be implanted intracranially (intracranial EEG) and/or located external to the head. Leads which are implanted intracranially may be placed on the surface of the brain and/or implanted within the brain tissue. Leads of the EEG monitoring device 216 may be configured, in use, to record neural activity at a neural structure in a brain of the subject. Where EEG is utilised for determining seizure activity data 106, the measurement unit 204 may also be used in conjunction with a signal generator (not shown) to measure electrode impedances.

The measurement unit 204 may be external to or integrated within the processing unit 202. Communication between the measurement unit 204 (and/or the signal generator) on the one hand and the I/O bus 214 on the other may be wired or may be via a wireless link, such as over inductive coupling, WiFi®, Bluetooth® or the like. Equally, communication between the measurement unit 204 and the one or more measurement devices 216, 218, 220, 222, 223, 224, 225 may be wired or may be via a wireless link such as those listed above.

Power may be supplied to some or all elements of the system 200 from at least one power source 224. The at least one power source 224 may comprise a battery such that elements of the system 200 can maintain power independent of mains power when implanted into a subject.

In some embodiments, some or all functions of the measurement unit 204 may be implemented using the processing unit 202, in which case, the one or more measurement devices 216, 218, 220, 222, 223, 224, 225 may be coupled directly to the I/O bus 214.

In some embodiments, the I/O bus 214 is configured for wired and/or wireless communications via a communications network, and the processing unit 202 is configured to cooperate with the I/O bus 214 to transmit or otherwise provide one or more temporal probabilistic models for determining a probability of a future seizure occurrence in each of a plurality of time windows for a particular subject to a remote server (not shown) or device (not shown). The device maybe a smart phone or watch associated with the subject for whom the one or more temporal probabilistic models have been generated. For example, the one or more temporal probabilistic models may be made available for the subject to download to a personal device from a website.

Memory 212 may further comprises computer executable code, (for example, a seizure forecasting module 212C), which when executed by the CPU 210, causes the processing unit 202 to determine an estimate of seizure probability in the subject for one or more of the plurality of time windows using the temporal probabilistic model. Memory 212 may further comprise computer executable code, (for example, an alert generation module 212C), which when executed by the CPU 210, causes the processing unit 202 to generate an alert based on the estimate of seizure probability in the subject for one or more of the plurality of time windows. For example, such alerts may include one or more of: a seizure occurrence probability, a seizure occurrence risk rating, information concerning the cause of risk elevation, a recommendation to take or change medication or therapeutic type or amount, a recommendation to administer stimulation or to alter one or more parameters of a therapeutic device for delivering stimulation to the subject.

In some embodiments, the processing unit 202 is configured to cooperate with the I/O bus 214 to transmit or otherwise provide the estimate of seizure probability in the subject for one or more of the plurality of time windows and/or an alert based on the estimate to a remote server (not shown) or device (not shown). In other embodiments, the generated one or more models may be stored on the subject's personal device, along with the seizure forecasting module 212B, and optionally, the alert generation module 212C.

The system 200 may further comprise one or more input device 208 and/or one or more output devices 206. The one or more input devices 208 may include, but are not limited to, one or more of a keyboard, mouse, touchpad and touchscreen. Examples of the one or more output devices 206 include displays, touchscreens, light indicators (LEDs), sound generators and haptic generators. Input and/or output devices 208, 206 may be configured to provide feedback (e.g. visual, auditory or haptic feedback) to a subject.

Feedback provided by the one or more output devices 206 may include information or an alert based on seizure occurrence likelihood in a subject, particularly a subject to which information recorded by the measurement unit 204 relates. Such information or alerts may include one or more of: a seizure occurrence probability, a seizure occurrence risk rating, information concerning the cause of risk elevation (e.g. increased heart rate, decreased HRV, sleep deprivation etc.), a recommendation to take or change medication or therapeutic type or amount, a recommendation to administer stimulation or to alter one or more parameters of a therapeutic device for delivering stimulation to the subject. Such information may be portrayed graphically or through the use of auditory or haptic feedback (as discussed above).

The one or more input devices 208 may enable the subject to acknowledge information and feedback provided via the one or more output devices 206 as well as input data into the system 200 which may then be used to improve forecasting of future seizures. This information may include data pertaining to the subject symptoms during a seizure. Such information may be used to verify or refute predictions previously made by the system 200. Other information may include seizure activity data 106 associated with the subject, which may be used to update the seizure activity data 106 stored in memory 212. In some embodiments, receipt of such seizure activity data 106 may instigate performance by the processing unit 202 of the method of FIG. 4 to generate one or more updated models temporal probabilistic models for the subject associated with the new seizure activity data 106.

The information or alerts based on the seizure occurrence likelihood in a subject may assist the subject, or an associate of the subject, such as the subject's career or clinician, to better manage their wellbeing, enabling pre-emptive administration of therapies and/or allow steps to ensure personal safety to be undertaken. The information, may for example, be used to schedule or alter administration of medicine or therapy.

To monitor the time of day for the purposes of forecasting seizure occurrence likelihood based on the circadian profile of a subject, the processing unit 202 may comprise a 12- or 24-hour clock operable to measure time.

The temperature sensor 223 may be provided to monitor temperature for the purpose of forecasting seizure occurrence likelihood based on a temperature based probability model associated with a subject.

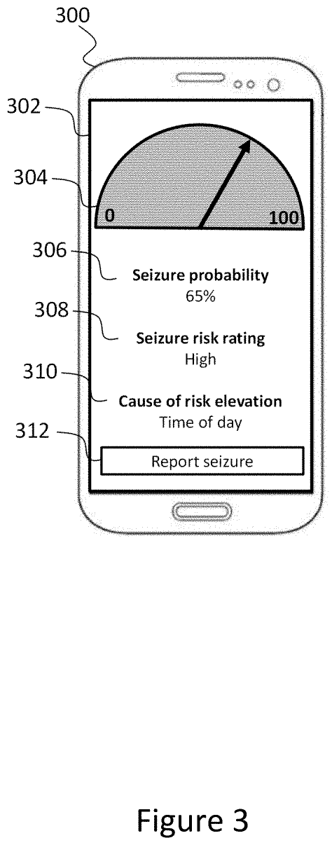

In some embodiments, some or all of the system 200 shown in FIG. 2 may be implemented using a smartphone or similar digital device (tablet, watch, computer etc.). FIG. 3 illustrates an exemplary device 300 upon which one or more units, components or modules of the system 200 may be implemented. The device 300 comprises a touchscreen display 302 which can function as both the input device 208 and output device 206 of FIG. 2. In the embodiment shown in FIG. 3, the touchscreen 302 provides a graphical illustration 304 of the probability of a seizure occurring, together with written information concerning seizure probability 306, a seizure risk rating 308 and information concerning the cause of elevation of risk 310 (as discussed above). In addition, a button 312 is provided on the touchscreen to enable a subject to input data concerning a seizure which has occurred. Auditory warnings may also be playable on the device 300.

In some embodiments, the one or more measurement devices 216, 218, 220, 222, 223, 224, 225 may be coupled to the device 300 via one or more wired or wireless links, in which case, the measurement unit 204 may be implemented using the device 300. Alternatively, the measurement unit 204 may be separate to the device 300, in which case, the measurement unit 204 itself may be wired or wirelessly coupled to the device 300.

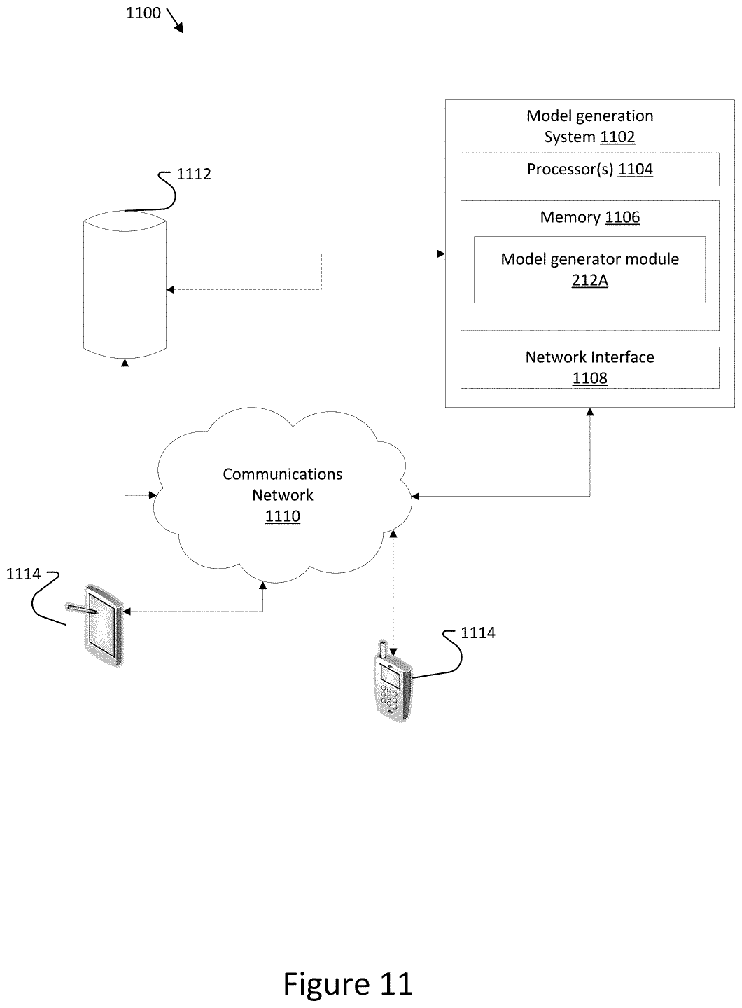

In some embodiments, as depicted in FIG. 11 there is shown a communications system 1100 comprising a model generation system 1102 for generating temporal probabilistic model(s) for forecasting future seizure activity in a subject having epilepsy, according to some embodiments. The model generation system 1102 comprises one or more processors 1104 and memory 1106. The processor(s) 1104 may include an integrated electronic circuit that performs the calculations such as a microprocessor, graphic processing unit, for example. In some embodiments, the model generation system 1102 may be implemented as a distributed system comprising multiple server systems configured to communicate over a network to provide the functionality of the model generation system 1102. Memory 1106 may comprise both volatile and non-volatile memory for storing executable program code, or data. Memory 1106 comprises program code which when executed by the processor(s) 1104, provides the various computational and data management capabilities of the model generation system 102. The block diagram of FIG. 11 illustrates some of the modules stored in memory 1106, which when executed by the processor(s) 1104 of the generation system 1102, performs the method 400 discussed below with reference to FIG. 4. For example, memory 1106 may comprise the temporal probabilistic model generator module 212A. The model generation system 1102 may further comprise a network interface 1108 to facilitate communications with components of the system 1102 across a communications network 1110, such as computer device(s) 1114, database 1112 and/or other servers. The network interface 1108 may comprise a combination of network interface hardware and network interface software suitable for establishing, maintaining and facilitating communication over a relevant communication channel.

The communications system 1100 may comprise the database 1112 for storing data used by the model generation system 1102 for generating temporal probabilistic model(s). The database 1112 may be implemented using a relational database or a non-relational database or a combination of a relational database and a NoSQL database. The model generation system 1102 may access the database 1102 directly or via the communications network 1110. The database 1112 may comprise historical data 102 associated with a plurality of subjects or patients. The historical data 102 may be recorded over a period of time and may comprise non-EEG physiological data 104, and seizure activity data 106. The database 1112 may by updated periodically or aperiodically to update historical data 102 associated with the respective subject.

Once the model generation system 1102 has generated one or more temporal probabilistic models for a particular subject, the model(s) may be deployed on a computer device 1114 for use by a patient. The computer device 1114 may be remote from the model generation system 1102. The computer device 1114 may be configured to download the generated temporal probabilistic model(s) from the model generation system 1102 via communications network 1110. For example, the computing device 1114 may be an end-user computing device such as a smart watch, a mobile device, a tablet device, a desktop computer, a laptop computer, etc.

In some embodiments, where updated historical data 102 is available to the model generation system 1102, the temporal probabilistic model generator module 212A of the model generation system 1102 may be configured to generate updated temporal probabilistic model(s) for forecasting future seizure activity in the subject, which may be provided as update(s) to the computing device 1114.

### Method

FIG. 4 is a process flow diagram of a method 400 of generating one or more temporal probabilistic models for forecasting future seizure activity in a subject having epilepsy, according to some embodiments. The method 400 may be executed by one or more processors of the processing unit 202 of the system 200 executing instructions stored in memory 212 of the system 200, such as the temporal probabilistic model generator module 212A, for example. In some embodiments, the method 400 may be performed by the model generation system 1102 executing instructions stored in memory 1106 of the system 1102, such as the temporal probabilistic model generator module 212A, for example.

At 402, the system 200, 1102 determines historical data associated with a subject experiencing epileptic events over a first time period. The historical data comprises non-EEG physiological data recorded over the first time period, and a time at which epileptic events occurred during the first time period. For example, the historical data may comprise a time at which each epileptic event occurred during the first time period.

In some embodiments, the non-EEG physiological data may comprise cardiac output recorded over the first time period. The cardiac output may comprise one or more of: (i) heart rate, and (ii) heart rate variability. For example, the non-EEG physiological data may comprise an electrocardiograph (ECG) and/or a photo-plethysmograph (PPG) signal received from the heart of the subject.

The non-EEG physiological data may comprise values of one or more variables of sleep recorded over the first time period. The non-EEG physiological data may comprise values of one or more variables of activity recorded over the first time period. For example, the non-EEG physiological data may comprise an actigraphy received from the subject.

The non-EEG physiological data may comprise values of one or more variables of oxygen saturation recorded over the first time period. The non-EEG physiological data may comprise values of one or more variables of electrodermal activity recorded over the first time period. The non-EEG physiological data may comprise values of one or more variables of respiratory rate recorded over the first time period.

In some embodiments, the historical data further comprises EEG physiological data. For example, the EEG physiological data may comprise an electroencephalography (EEG) signal received from the brain of the subject.

At 404, the system 200, 1102 extracts from the non-EEG physiological data, one or more temporal models indicative of a subject specific cycle, or repeating cycling. For example, a temporal model may have a circadian, an ultradian, or an infradian period. In a typical case, the system may be expected to extract a first temporal model indicate of a 24-hour cycle and second temporal model indicate of an about-weekly (5-10 days) cycle.

In some embodiments, the system 200, 1102 transforms the non-EEG physiological data into the time-frequency domain to detect the one or more temporal models, that is, cycles at a given time period. For example, in some embodiments, wavelet decomposition is used to identify the subject specific cycles.

In some embodiments, before using the wavelet decomposition, the EEG physiological data, for example in the form of a continuous signal, may be downsampled and linear interpolation may be performed on the downsampled data to compensate for missing segments.

The system 200, 1102 may be configured to determine local maxima (peaks) in the wavelet spectrum by comparing neighbouring values and to determine local maxima having a confidence level above the global significance level as being significant cycle periods, for example, by using a time-averaged significance test.

At 406, the system 200, 1102 generates one or more temporal probabilistic models based on the respective one or more temporal models, the non-EEG physiological data, and the times at which each epileptic event occurred, wherein each temporal probabilistic model is representative of a probability of a future seizure activity in each of a plurality of time windows.

In some embodiments, for one or more of the temporal models, the system 200, 1102 filters the signal indicative of the non-EEG physiological data at the period of the temporal models, i.e., at the cycle frequencies identified from the wavelet decomposition. The non-EEG physiological data may be filtered, for example, using one or more bandpass filters, into one or more component frequencies corresponding to the one or more temporal models. The system may downsample and interpolate the non-EEG physiological data before filtering the signal. For example, the bandpass filter applied at each significant cycle may be a second-order zero-phase Butterworth bandpass filter with cutoff frequencies at +/−33.3% of the cycle frequency.

The system 200, 1102 determines a phase of the filtered non-EEG physiological data. For example, the Hilbert transform may be used to estimate the continuous phase of each bandpass filtered signal. The system 200, 1102 maps the times of the epileptic events to the phase of each of the filtered non-EEG physiological data to generate the one or more temporal probabilistic models. In other words, for each of the filtered signals, the system 200 determines a phase of the filtered signal at times of past epileptic event occurrences to determine the one or more temporal probabilistic models. The one or more temporal probabilistic models are indicative of a probability of a seizure occurring with respect to the phase of the non-EEG physiological data signal.

In some embodiments, the system 200, 1102 combines a plurality of the temporal-probabilistic models into a single forecast of future seizure activity likelihood. For example, the system 200 may combine the plurality of the temporal-probabilistic models using any suitable weighted approach, such as simple multiplication, Bayes rule, logistic regression, etc. The system 200, 1102 may select temporal-probabilistic models to be combined by determining whether a candidate temporal-probabilistic model is sufficiently significant or meets a specific requirement. For example, for a candidate to be considered significant, it may have a non-uniform distribution.

At 408, the system 200, 1102 provides, for example as an output, the one or more temporal probabilistic models for determining an estimate of seizure probability in the subject for one or more of the plurality of time windows. In some embodiments, the system 200, 1102 may transmit or otherwise provide the one or more temporal probabilistic models to a remote server or device, such as computer device 1114, for use by the server or device to determine an estimate of seizure probability in the subject for one or more of the plurality of time windows.

At 410, in some embodiments, the system 200, 1102 determines an estimate of seizure probability in the subject for one or more of the plurality of time windows using the one or more temporal probabilistic models.

At 412, the system 200 may output, for example to an output device 206 of the system 200, an alert based on the estimate of seizure probability in the subject for one or more of the plurality of time windows.

In some embodiments, the method may iteratively update the one or more temporal probabilistic models for the subject based on updated historical data received and associated with the subject. For example, the system 200, 1102 may receive or access, for example, in database 1112, updated historical data associated with the subject over a second time period. The second time period may include the first time period. The updated historical data may comprise updated non-EEG physiological data recorded over a second time period and/or a time at which epileptic events occurred during the second time period. For example, the updated historical data may comprise a time at which each epileptic event occurred during the second time period. In response to receiving the updated historical data, the system 200, 1102 may determine one or more updated temporal probabilistic models for determining an estimate of seizure probability in the subject for one or more of the plurality of time windows.

For example, where the updated historical data includes updated non-EEG physiological data, the system 200, 1102 may extract from the updated non-EEG physiological data, one or more updated temporal models indicative of the subject specific cycle.

The system 200, 1102 may generate one or more updated temporal probabilistic models the respective one or more updated temporal models, the updated non-EEG physiological data, and the times at which each epileptic event occurred during the first or second periods of time, wherein each updated temporal probabilistic model is representative of a probability of future seizure activity in each of a plurality of time windows. The system 200, 1102 may then provide the one or more updated temporal probabilistic models for determining an estimate of seizure probability in the subject for one or more of the plurality of time windows.

Study Establishing that Cardiac Output Shows Multiday Rhythms Akin to Cycles of Cortical Excitability, which Modulate an Individual's Likelihood of Having a Seizure.

Data used to develop some of the methods described herein was collected from an observational cohort study. A dataset of historical data (dataset A) including cardiac output and epileptic activity was monitored using long-term mobile seizure diaries and a wearable smartwatch. Findings were validated using a retrospective dataset (dataset B), which comprises one week of recordings from subjects undergoing ambulatory video/EEG/ECG for epilepsy diagnostic testing.

These two datasets provided complementary information. Mobile data provides an approximation of the periodic occurrence of epileptic seizures. Similarly, wearable devices for heart rate monitoring have lower resolution than ECG. However, these limitations are balanced by the ability to collect long-term recordings over months to years. On the other hand, ambulatory video/EEG/ECG electrodes enable extremely accurate quantification of both heart rate and rates of epileptic activity/seizures, with the trade-off of a limited duration (7-14 days) over which to record cycles.

The study was devised to measure cycles of seizure likelihood from seizure diaries, identify corresponding cycles from non-invasively measured physiological signals, and quantify the relationship between physiological cycles and seizure occurrence. The study was approved by the St Vincent's Hospital Human Research Ethics Committee (HREC 009.19) and the first enrolment was in August 2019.

The study recruitment target was 30 participants over the first year. Participants were over 18, with a confirmed epilepsy diagnosis and uncontrolled or partially controlled seizures. Continuous data were collected via mobile and wearable devices for at least 2 months and up to 2 years. Participants wore a smartwatch and manually reported seizure times in a freely available mobile diary app. The smartwatch continuously measured participants' heart rates (via photoplethysmograph) at 5 s resolution. The smartwatch also monitored estimated sleep stage (wake, REM, light and deep sleep), and step count per hour. The cohort had 45 participants (15 male) with a cumulative total of 106280 (M=2872, SD=2487) hours of continuous heart rate recorded and over 4289 (M=134, SD=104) nights of sleep scoring. Mean device adherence (at least one recording every hour) among participants was 66% (SD=36%). The total diary duration across participants was 57 years (between 40 and 3367 days per person). Participant diaries include over 3179 (M=79, SD=105) seizures with 1455 (M=39, SD=60) seizures reported during the wearable monitoring period.

Primary study outcome measures were the strength of physiological cycles and the strength of co-modulation between physiological cycles and seizure occurrence.

### Multi-Day Heart Rate Cycles

To evaluate the strength of heart rate cycles, participants were required to have at least 2 months of recordings, with over 80% adherence (i.e. less than 20% missing data).

Cycles were measured at different periods using a wavelet transform approach that has been previously proposed for the detection of multiday cycles of epileptic activity, as disclosed in “Baud”. The continuous heart rate signal was first downsampled to one timestamp every minute. Linear interpolation was performed for up to 1 hr either side of a missing segment. Longer recording gaps of up to 3 days were interpolated with a straight line at the average value of all the data. This method of interpolation detected 93.5% of true cycle periods whilst minimising false cycle detections.

A Morlet wavelet decomposition with increased spacing was used on standardised data segments longer than one month to compute the power at different scales (cycle periods). The scales were every 1.2 hours between 2.4 and 31.2 hours, every 2.4 hours between 33.6 and 48 hours, every 4.8 hours between 52.8 and 4 days and every 12 hours between 5 days up to a maximum period of half the recording duration.

The data were then represented as a global wavelet spectrum of power (the product of the average of the square of absolute value of complex wavelet coefficients and the variance of the time series) for each individual scale.

Local maxima (peaks) in the wavelet spectrum were found by comparing neighbouring values. Local maxima above the global significance (95% confidence) level were determined to be significant heart rate cycle periods using a time-averaged significance test. A suitable time-averaged significance test is described in Torrence C, Compo G P. A practical guide to wavelet analysis. Bulletin of the American Meteorological society 1998; 79: 61-78.

Heart rate cycle periods were considered to be circadian, weekly or monthly if they were within +−33.3% of 24 hours, 7 days or 30 days, respectively. Multiday cycles were any other periods longer than 32 hours and ultradian cycles were any other periods shorter than 16 hours.

### Relationship Between Seizures and Heart Rate Cycles

To evaluate the strength of seizure phase locking to heart rate cycles, participants were required to have at least 10 reported seizures.

After downsampling and interpolation, the standardised heart rate signal was bandpass filtered into distinct component frequencies matching the significant cycle frequencies (identified from wavelet decomposition). The bandpass filter applied at each significant cycle was a second-order zero-phase Butterworth bandpass filter with cutoff frequencies at +/−33.3% of the cycle frequency. For instance, someone with significant cycles (wavelet spectrum peaks) at 24 hours, 9 days, and 30 days would have three bandpass filters applied with cutoff frequencies of 16-32 hours, 6-12 days and 20-40 days, respectively. These cutoff frequencies were chosen to account for changes in the cycle period over the recording time.

The continuous phase of each bandpass filtered signal was estimated using the Hilbert transform. The times of seizure occurrence were mapped to the estimated phase of heart rate cycles. Seizure phases were then binned into 24 (circadian cycles) or 18 (all other periods) equal sized bins (ranging from 0 to 2 pi) to produce a phase distribution. The phase distributions were used to determine whether seizures were phase-locked to the underlying heart rate cycle, indicating co-modulation (direct or indirect) between seizure occurrence and heart rate. Seizure phase locking was quantified by the synchronisation index (SI, also known as a phase-locking value or R-value).

For each patient, any given seizure time can be expressed as a phase of a cycle with some arbitrary period. The mean resultant length, R, of phase of all seizures for a patient can be determined to thereby quantify the seizure phase locking. The R-value is also known as the synchronisation index (SI) or phase-locking value. The mean resultant length, R, which is the mean phase coherence of an angular distribution, can be calculated using the following equation:

$$R = \frac{1}{N} {\sum }_{n = 1}^{N} {e}^{i θ} = 1 - C V$$

where θ is the relative phase, N is the number of samples of the data set, CV denotes the circular variance of an angular distribution obtained by transforming the relative phase angles onto the unit circle in the complex plane. R quantifies the degree of phase locking to a cycle with a particular period. R has values in [0 1], R reaches the value 1 if and only if the condition of strict phase locking is obeyed—perfect alignment to an underlying cycle. That is, all seizures occur at precisely the same phase of a given cycle (for example, at the exact peak of an underlying heart rate cycle). For a uniform distribution of phases, for example, in completely unsynchronized series of seizures, (i.e. no relationship), R=0.

The angle, or direction of SI indicates the preferred phase of seizure occurrence (the circular mean of the distribution), for instance seizures could be more likely near the peak or trough of average heart rate cycles. The Omnibus (or Hodges-Ajne) test was used to determine whether seizures were significantly (p<0.05) phase-locked to the heart rate cycle by testing the null hypothesis that the phase distribution was uniform.

### Dataset B—Ambulatory EEG/ECG

Retrospective analysis of EEG/ECG used a database of people undergoing at-home, ambulatory video-EEG/ECG diagnostic testing for epilepsy. The study was approved by the St Vincent's Hospital Human Research Ethics Committee (LRR 165/19).

This study used 30 records of at least 8 days duration (range 8-14 days). Only continuous ECG data were used, combined with event labels derived from video-EEG. Events were labelled using computer-assisted review, whereby event detection was first performed by a machine learning algorithm. Suspect events were then reviewed and confirmed by expert neurophysiology and neurology review. Event labels consisted of algorithm detections, confirmed epileptic activity and diary labels.

To compare multiday ECG recordings to the heart rate recorded by smartwatches, r-peak detection was performed using the method described by Christov et al. (2004), Real time electrocardiogram QRS detection using combined adaptive threshold. Biomedical engineering online 2004; 3: 28, the entire content of which is incorporated herein by reference.

Following peak detection, a number of beats per minute, with a 5 s sliding window were computed. Heart rate variability (HRV) was also investigated by computing the variance of the peak-to-peak intervals within a 1 m window, updated every 5 s. To investigate circadian cycles, the heart rate and HRV signals were first downsampled to one timestamp every hour, then filtered using a bandpass filter capturing cycles between 16 and 32 hours. Signal phase was computed using the Hilbert transform and phase locking of epileptiform activity was measured using the SI.

Given the limited duration of ECG studies, it was not possible to determine whether a weekly cycle was present in the heart signal. Instead, whether significant linear or quadratic trends were observed in the smoothed heart rate signal was identified. Heart rate was smoothed using a 1-day moving average filter. Linear regression was then performed for a linear model, and a quadratic model to describe heart rate with respect to time, t. The rationale for this approach was that the ECG might be recorded around the peak or trough of an about-weekly cycle (showing a curved/quadratic trend), or on the rising/falling slope of a cycle (showing a linear trend). The linear model is given as: heart rate=at +c, for some constants a and c. The quadratic model is given as: heart rate=at²+bt+c. Linear regression was used to estimate the coefficients, a, b, and c and the p-values for each coefficient, to determine whether time features significantly correlated with heart rate (p<0.05). r\hat{ }2, or the proportion of variability in the heart rate signal described by either model was also estimated.

### Results

Out of 39 participants, there were 18 with at least two months of data (range 2.2 to 11.8 months) and 80% adherence. Mean adherence of wearable devices among eligible participants was 95%. Among these 18 participants, 12 had recorded at least 10 seizures during the wearable recording time and were therefore eligible for further seizure analysis.

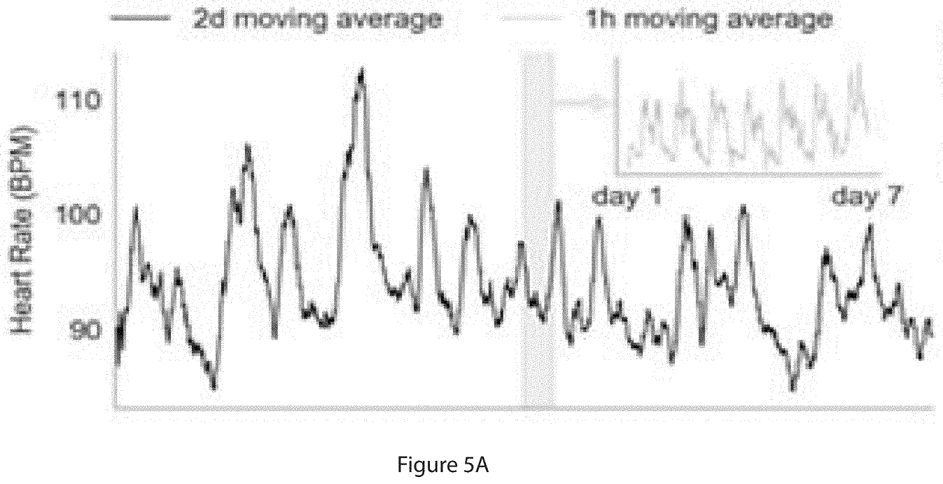

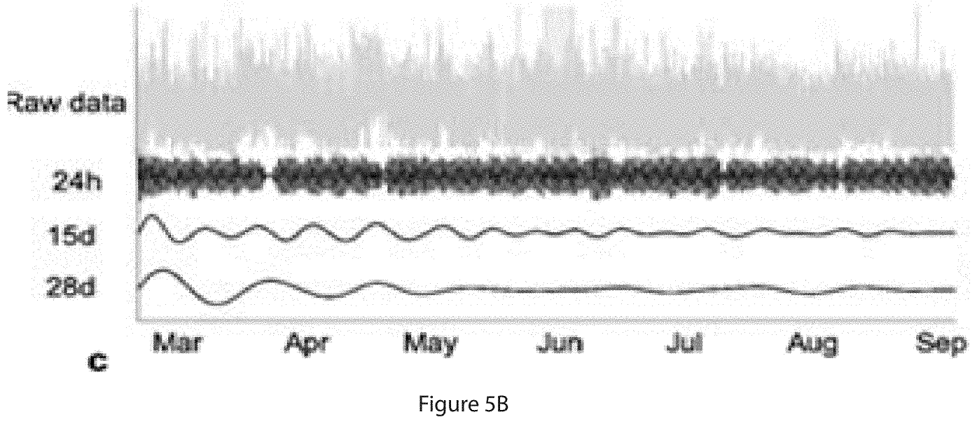

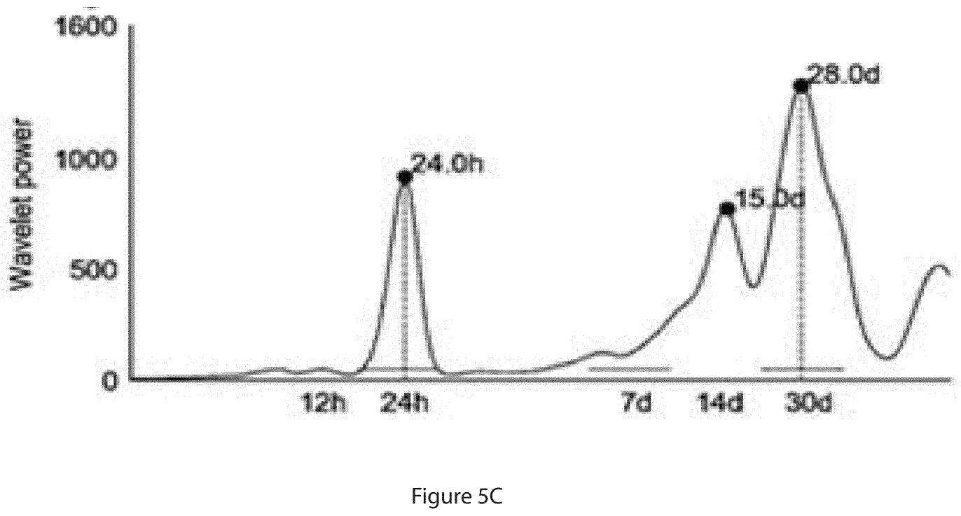

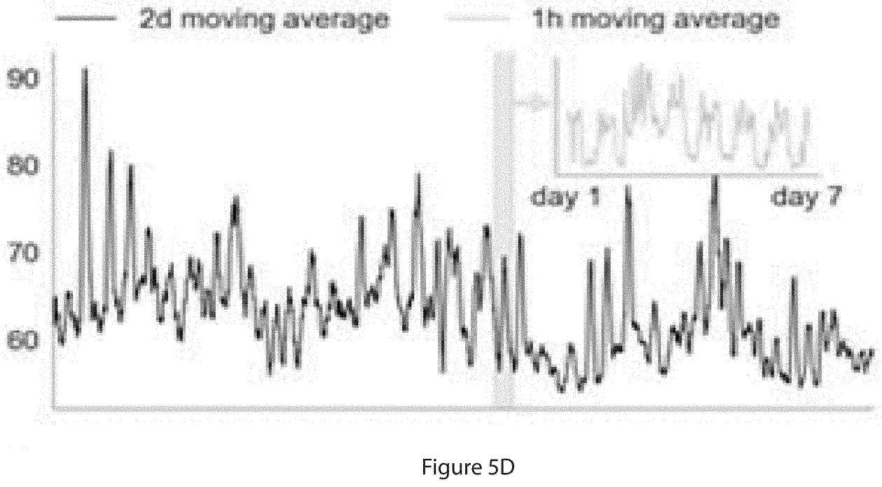

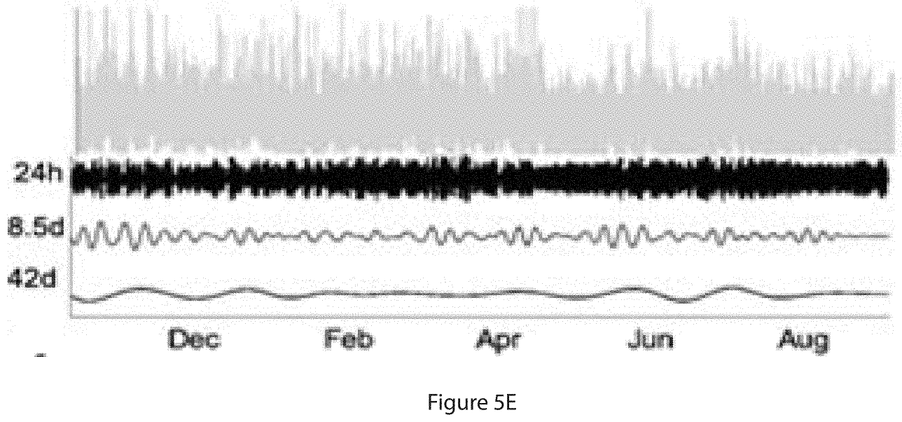

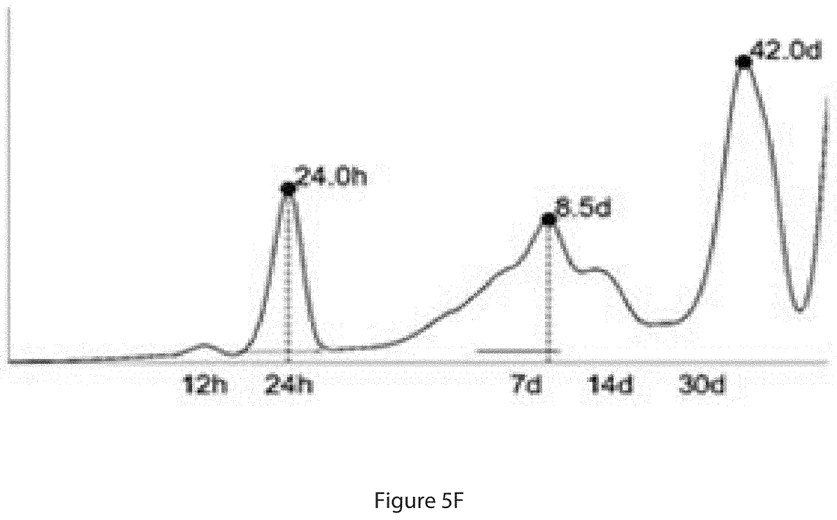

The data was analysed to first investigate whether multiday cycles of average heart rate existed. Cycles at multiple periods (scales) were detected using a wavelet transform. FIGS. 5A and 5D depict plots of heart rate cycles for two individuals showing multiday cycles. The heart rate (y-axis) is smoothed with a 2-day moving average filter. Insets show circadian rhythms of heart rate. FIGS. 5B and 5E depict bandpass filtered heart rate signals for different cycles (corresponding to FIGS. 5C and 5F). FIGS. 5C and 5F depict wavelet power spectrum for different scales (x-axis). Significant cycle periods are labelled. Multiday rhythms of heart rate were evident over about-weekly (FIG. 5E) and about-fortnightly (FIG. 5B) periods.

Wavelet analysis confirmed significant cycles at daily (24 h), fortnightly (15 d) and monthly (28 d) periods for 51, and daily (24 h), weekly (8.5 d) and slower (48.5 d) periods for S2. These individual examples provide striking evidence that multiday cycles in cardiac output exist over weekly and monthly timescales. Cycles can be seen from visual inspection of average heart rate and were robust over months.

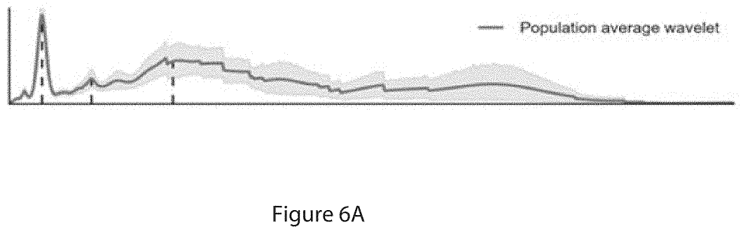

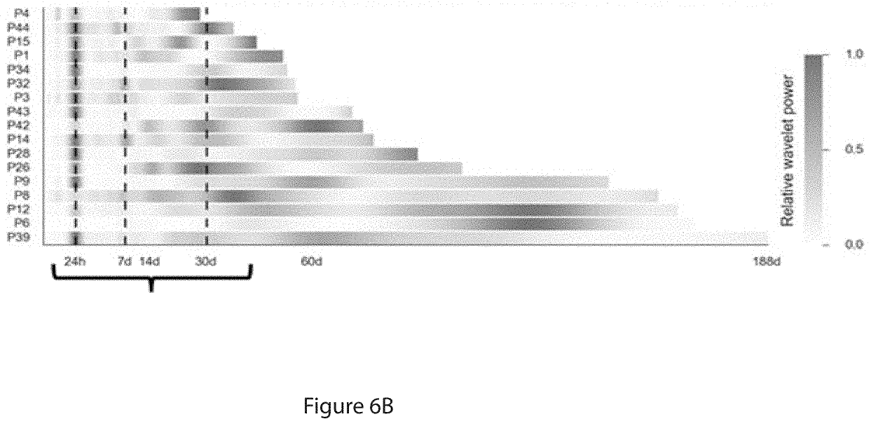

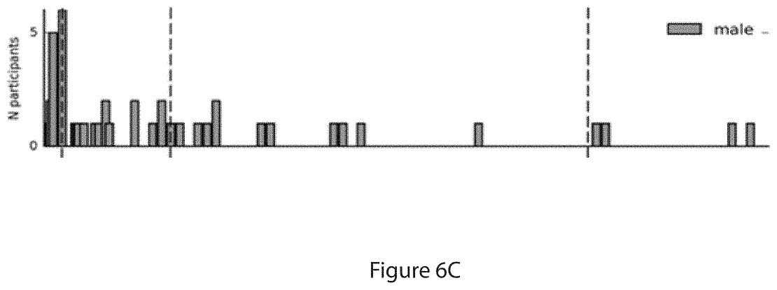

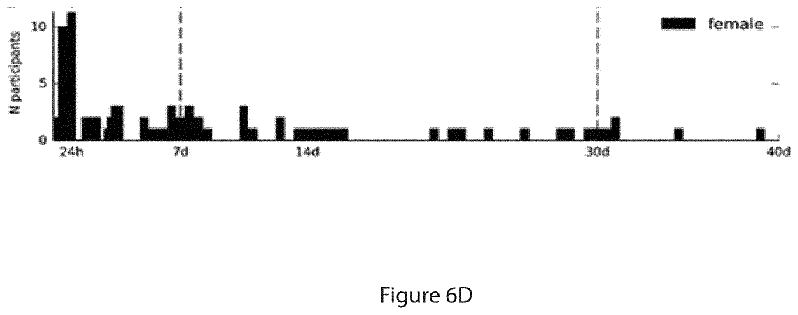

FIGS. 6A to 6D, plots of a distribution of heart rate cycles over time, show the prevalence of multiday heart rate cycles across the cohort. FIG. 6A illustrates cycle strength (expressed as the normalised wavelet power, y-axis) for different periods (x-axis) averaged across the cohort; FIG. 6B depicts a raster plot showing cycle strength for each individual (y-axis) at different periods (logarithmic scale); FIGS. 6C and 6D depict a number of people (y-axis) with significant cycles at different periods (x-axis, logarithmic scale) for men and women, respectively. Note that in FIGS. 6C and 6D the x-axis (up to 40 days) is a subset of the x-axis (up to 188 days) in FIGS. 6A and 6B.

It can be seen that cycles exist at weekly, fortnightly, monthly and longer timescales. The average cycle strength across the cohort showed a clear peak at 24-hours, with significant circadian cycles detected for 100% of the cohort. Further, the peaks in cycle strength at 7-days, 14-days and 30-days, demonstrate that subsets of people have similar cardiac cycles at these periods.

Notably, multiday cardiac cycles were as prevalent as circadian cycles (found in 100% of the cohort), albeit with a greater degree of individual variability. To account for this variability, we considered specific cycle periods to encompass a band of +/−30% around a central frequency. For instance, about-weekly cycles were characterised as periods of 5-10 days. Strikingly, 18 people (100%) showed an about-weekly cycle and 14 people (78%) had about-monthly cycles. Apart from these periods, 17 people (94%) had a multiday cycle (>32 hours) other than weekly or monthly and 15 people (83%) had a shorter cycle (<=32 hours) other than circadian.

The distributions of cycles were overall similar for men as women (FIGS. 6c and 6d). Everyone showed daily and about-weekly cycles and of the people with about-monthly cycles, 4 were men (57% of all eligible male participants) and 10 were women (91% of females). Note that only three (17%) of about-weekly cycles were found at precisely 7-days, suggesting that, in general, cycles were not related to a particular day of the week. Interestingly, this was consistent with a previous finding that 21% of people have a precise 7-day seizure cycle [Karoly et al., 2018]; however, our results found that only one person (6%) had seizures significantly locked onto their 7-day heart rate cycle.

### Multi-Day Heart Rate Cycles Modulate Seizure Risk

The study involved investigating whether multiday cardiac cycles and seizure likelihood were co-modulated by analysing whether self-reported seizure times were significantly phase-locked to the underlying heart rate cycles (quantified by a synchronization index, SI).

Individuals with more than 10 seizures were included in the analysis, resulting in a total of 12 people. Of these, 11 of 12 (92%) people had seizures significantly locked onto at least one heart rate cycle. Among participants with observed seizure modulation, 73% had both circadian and multiday cycles co-modulated with seizure risk, 82% had seizures significantly locked onto a multiday cycle and 91% had seizures significantly locked onto a circadian or ultradian cycle.

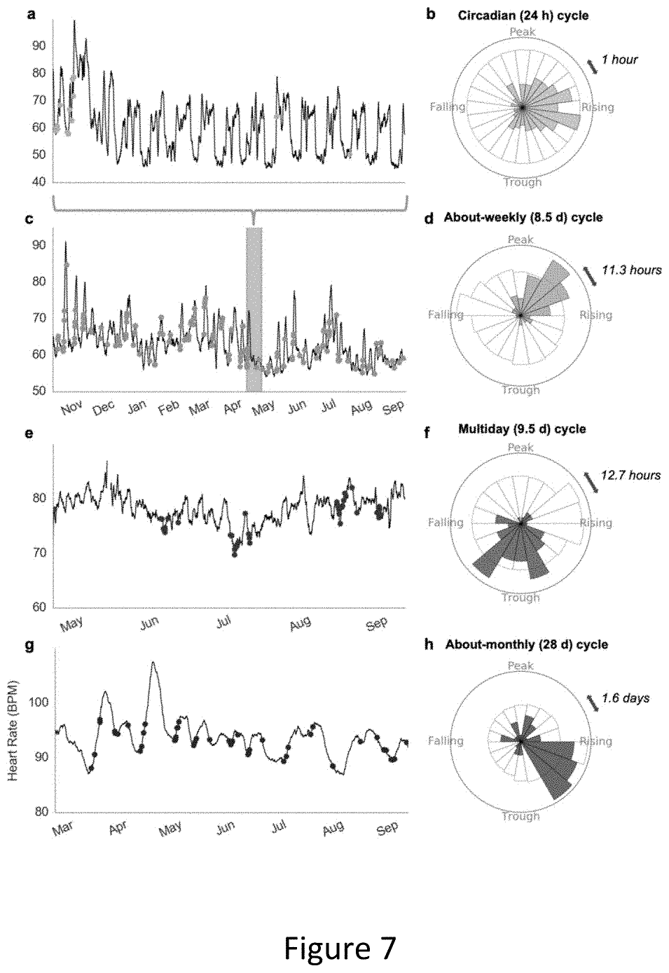

FIGS. 7A to 7H show examples (for three individuals: P12, P34, P43) of the occurrence of seizures with respect to multiday cycles of heart rate. FIGS. 7A, 7C, 7E and 7G show heart rate (y-axis) and self-reported seizures (dots) for three different participants. The corresponding circular histograms of FIGS. 7B, 7D, 7F and 7H show the phase distribution of seizures with respect to a significant heart rate cycle. Note that multiday histogram bins have the same phase width (2*pi/18) although this corresponds to different durations (labelled). The circadian histogram (b) bins have width of 1 h (2*pi/24). FIGS. 7A and 7C show a circadian and about-weekly cycle, respectively, for P12 and FIGS. 7B and 7C show the corresponding phase distributions. FIGS. 7E and 7F show a multiday (fortnightly) cycle for P42 and corresponding phase distribution. FIGS. 7G and 7H show an about-monthly cycle for P3 and corresponding phase distribution. Heart rate is shown after applying a moving average (MA) filter to highlight cycles (FIG. 7A uses a 1-hour MA, FIGS. 7C and 7E use a 2-day MA and FIG. 7G uses a 7-day MA).

It can be seen that different individuals' seizures were strongly synchronised with their unique multiday heart rate cycle. Often synchronisation was stronger for multiday cycles than circadian cycles; for instance P12 showed SI=0.61 for their 8.5 day cycle and SI=0.37 for their circadian cycle.

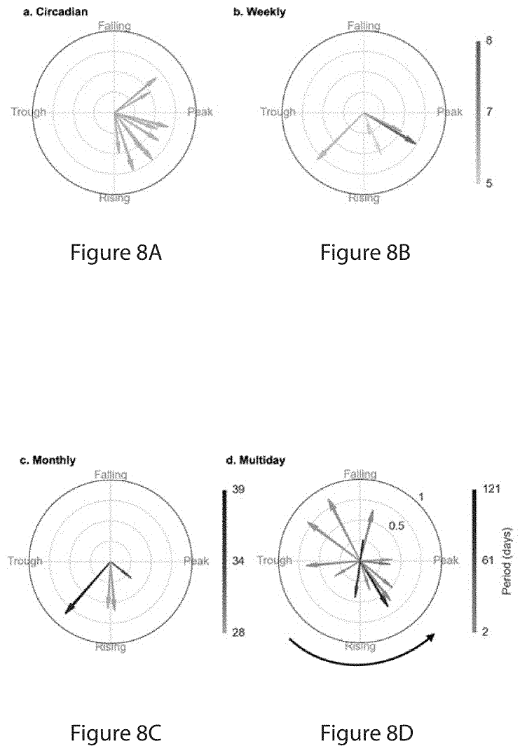

Phase locking of seizures to heart rate cycles is shown in FIGS. 8A to 8D including the SI values across the cohort for different cycles. Each figure shows individuals (arrows) with significant phase locking of seizure occurrence to their heart rate cycle. The length of the arrows indicates the strength of phase locking (radial axis, between 0 and 1), while the direction indicates the preferred phase (polar axis). FIG. 8A shows a circadian cycle: all periods were 24 hours; FIG. 8B shows an about-weekly cycle: 5-8 day periods; FIG. 8C shows an about-monthly cycle: 27-30 day periods; and FIG. 8D shows a multiday cycle: 1-121 day periods. It can clearly be seen that seizures preferentially occurred on the rising phase of multiday cycles (weekly, monthly, and other multiday periods, FIGS. 8B-D) for most people. In other words, the gradually increasing heart rate is linked to increased seizure likelihood. On the other hand, seizure occurrence with respect to circadian heart rate cycles was equally seen during, before and after the cycle peak (FIG. 8A).

### Circadian Cycles and Weekly Trends in Continuous EEG/ECG

The weekly modulation of heart rate, as well as the cyclic co-modulation of heart rate, and epileptic activity was validated using continuous EEG/ECG records from a cohort of patients undergoing diagnostic monitoring for epilepsy.

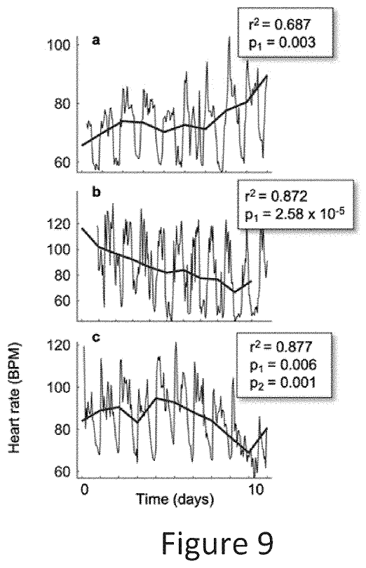

Whether significant weekly trends were detected from ECG was first investigated. FIG. 9A to 9C show example cases where different weekly trends were observed, identified from ambulatory ECG monitoring. Thin black lines show average heart rate (y-axis) and thick lines show smoothed data (2-day moving average). Significant trends were identified from the smoothed data using logistic regression. FIGS. 9A and 9B show increasing and decreasing linear trends. FIG. 9C shoes a curved (quadratic) trend. Insets show r-squared values and p-values for the linear (p_1) and quadratic (p_2) components.

Across the EEG-ECG cohort, significant trends were observed in 15 out of 29 (52%) ECG studies, with 9 studies showing a linear trend and 11 showing a curved trend. For people with a significant trend (p<0.05) the r-squared values ranged from 0.44 to 0.95 (mean 0.69). Although it was not possible to determine whether these trends were associated with about-weekly cycles, these findings support the observation that individuals' average heart rate is slowly modulated over weekly time scales.

The cohort of EEG-ECG studies were also used to determine the relationship between epileptic events and circadian cycles of heart rate and HRV. Most of the cohort showed significant phase locking of epileptic events to their circadian cycles of heart rate and HRV (see Table I below). For all event detections, 88% of the cohort were phase locked to heart rate cycles, dropping to 65% for confirmed epileptic discharges and 64% for self-reported events. Note that this decrease in people with significant phase locking is most likely due to lower event numbers. Over the one week EEG-ECG studies, significant phase locking was also observed with respect to clock time. Across the cohort significant differences in SI between heart rate or HRV and clock time were not found for any event type (p>0.05 using a paired t-test).

TABLE I

| | | Significant SI | | | Mean SI | | | | |
|---|---|---|---|---|---|---|---|---|---|
| Event type | Eligible | Time | Heart rate | HRV | Time | Heart rate | p-value | HRV | p-value |
| Self-report | 14 | 9 | 9 | 5 | 0.68 | 0.54 | 0.134 | 0.55 | 0.122 |
| Epileptic | 27 | 18 | 17 | 18 | 0.60 | 0.63 | 0.810 | 0.45 | 0.204 |
| Event | 26 | 21 | 23 | 21 | 0.54 | 0.48 | 0.105 | 0.44 | 0.066 |
| detections | | | | | | | | | |

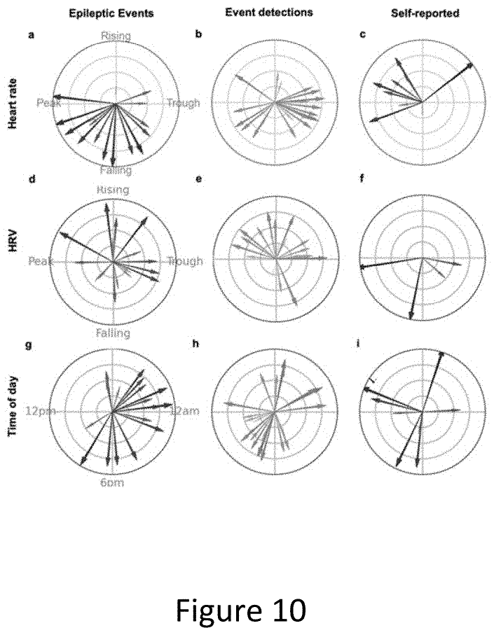

FIGS. 10A to 10I show the phase locking of epileptic activity to circadian cycles of heart rate, HRV and time of day. Each figure shows individuals (arrows) with significant phase locking of events to their circadian cycle of heart rate (FIGS. 10A to 10C), HRV (FIGS. 10D to 10F) or time of day (FIGS. 10G to 10I). The length of the arrows indicates the strength of phase locking (radial axis, between 0 and 1), while the direction indicates the preferred phase/time (polar axis). For the epileptic events depicted in FIGS. 10A, 10D and 10G, epileptiform discharges were detected on EEG and confirmed by expert neurophysiology review. For FIGS. 10B, 10E and 10H, the candidate epileptic discharges detected by an AI algorithm [Clark et al. 2019]. For FIGS. 10A 10F and 10I, seizures were self-reported during ambulatory video-EEG-ECG monitoring via a mobile app.

Confirmed epileptic events were more prevalent after the peak of the circadian heart rate, during the decreasing phase of the cycle. This finding is consistent with increased nocturnal occurrence of epileptiform discharges, as heart rate is decreased during sleep. Self-reported events occurred mainly before peak heart rate, during the increasing phase of the cycle, possibly reflecting a bias towards reporting more events during the day. The relationship between epileptic activity and circadian cycles of HRV was different to heart rate. Peak occurrences of epileptic events were distributed across both increasing and decreasing phase of the HRV cycle, while event detections were mainly concentrated during the rising phase.

The results of the study demonstrate that multiday cycles of heart rate were highly prevalent and co-modulated with already established multiday cycles of seizure likelihood. Heart rate cycles were especially prevalent at about-weekly timescales (5-10 days), although fortnightly and monthly cycles were also found across the cohort. Cycle periods were patient-specific and were not linked to day of the week or clear environmental cues, and showed approximately similar distributions for men and women.

These results confirm that cardiac cycles exhibit strikingly similar features to multiday cycles documented in epileptic brain activity. Furthermore, they demonstrate that the clear preference for seizures to occur is on the rising phase of multiday heart rate cycles (i.e. during increasing heart rate), which is also a property of epileptic rhythms.

The study also found weekly and monthly cycles across both men and women. The study established weekly trends using clinical standard heart rate (measured from continuous ECG) and demonstrated phase locking between epileptic activity and circadian heart rate cycles. About-weekly cycles were the most common periodicity observed in heart rate cycles presented in this study (FIG. 6). The current study also demonstrated trends (linear increase/decrease and parabolic) in heart rate measured from one week ECG monitoring, although weekly cycles could not be captured from the limited duration recording (FIG. 9).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method comprising:
determining historical data associated with a subject experiencing epileptic events over a first time period, the historical data comprising non-EEG physiological data recorded over the first time period, and a time at which epileptic events occurred during the first time period;
extracting from the non-EEG physiological data, one or more temporal models indicative of a subject specific cycle;
generating one or more temporal probabilistic models based on the respective one or more temporal models, the non-EEG physiological data, and the times at which
epileptic event occurred, wherein each temporal probabilistic model is representative of a probability of future seizure activity in each of a plurality of time windows; and
providing the one or more temporal probabilistic models for determining an estimate of seizure probability in the subject for one or more of the plurality of time windows.

2. The method of claim 1, further comprising determining the estimate of seizure probability in the subject for one or more of the plurality of time windows using the one or more temporal probabilistic models.

3. The method of claim 2, further comprising outputting an alert based on the estimate of seizure probability in the subject for one or more of the plurality of time windows.

4. The method of claim 3, wherein the alert comprises one or more of:
(i) the estimate of seizure probability in the subject for one or more of the plurality of time windows;
(ii) a seizure occurrence risk rating;
(iii) information concerning a cause of risk elevation;
(iv) a recommendation to take or modify change medication or therapy; and
(v) a recommendation to alter one or more parameters of a therapeutic device for delivering stimulation to the subject.

5. The method of claim 2, comprising scheduling administration of medication based on the estimate of seizure probability in the subject.

6. The method of claim 1, wherein generating the one or more temporal probabilistic models comprises:
filtering the non-EEG physiological data into one or more component frequencies corresponding to the one or more temporal models to produce one or more respective filtered non-EEG physiological data;
determining a phase of each of the filtered non-EEG physiological data; and
mapping the times of the epileptic events to the phase of each of the filtered non-EEG physiological data.

7. The method of claim 1, wherein the non-EEG physiological data comprises cardiac output recorded over the first time period.

8. The method of claim 6, wherein the cardiac output comprises one or more of: (i) heart rate, and (ii) heart rate variability.

9. The method of claim 1, wherein the non-EEG physiological data comprises values of one or more variables of sleep recorded over the first time period.

10. The method of claim 9, wherein the one or more sleep variables comprises one or more of: historical times of first waking and sleeping, time of hours awake over a previous time period, time of hours asleep over a previous time period, and sleep depth.

11. The method of claim 1, wherein the non-EEG physiological data comprises values of one or more variables of activity recorded over the first time period.

12. The method of claim 1, wherein the non-EEG physiological data comprises one or more of:
(i) values of one or more variables of oxygen saturation recorded over the first time period;
(ii) values of one or more variables of electrodermal activity recorded over the first time period;
(iii) values of one or more variables of respiratory rate recorded over the first time period; and
(iv) values of one or more variables of skin temperature recorded over the first time period.

13. The method of claim 1, wherein the epileptic events are associated with abnormalities in the non-EEG physiological data.

14. The method of claim 1, wherein the non-EEG physiological data comprises: (i) an electrocardiograph (ECG) received from the heart of the subject or (ii) a photo-plethysmograph signal received from the heart of the subject.

15. (canceled)

16. The method of claim 1, wherein the non-EEG physiological data comprises an actigraphy received from the subject.

17. The method of claim 1, further comprising:
determining updated historical data associated with the subject experiencing epileptic events over a second time period, the updated historical data comprising updated non-EEG physiological data recorded over a second time period and a time at which epileptic events occurred during the second time period;
extracting from the updated non-EEG physiological data, one or more updated temporal models indicative of the subject specific cycle;
generating one or more updated temporal probabilistic models based on the respective one or more updated temporal models, the updated non-EEG physiological data, and the times at which each epileptic event occurred during the second period of time, wherein each updated temporal probabilistic model is representative of a probability of future seizure activity in each of a plurality of time windows; and
providing the one or more updated temporal probabilistic models for determining an estimate of seizure probability in the subject for one or more of the plurality of time windows.

18. The method of claim 17, further comprising receiving new seizure data for the second period of time and responsive to receiving the new seizure data, determining the updated historical data.

19. The method of claim 17, wherein the second time period includes the first time period.

20. An seizure forecasting system comprising:
one or more processors; and
memory comprising computer executable instructions, which when executed by the one or more processors, is configured to:
determine historical data associated with a subject experiencing epileptic events over a first time period, the historical data comprising non-EEG physiological data recorded over the first time period, and a time at which epileptic events occurred during the first time period;
extract from the non-EEG physiological data, one or more temporal models indicative of a subject specific cycle;
generate one or more temporal probabilistic models based on the respective one or more temporal models, the non-EEG physiological data, and the times at which epileptic event occurred, wherein each temporal probabilistic model is representative of a probability of future seizure activity in each of a plurality of time windows; and
provide the one or more temporal probabilistic models for determining an estimate of seizure probability in the subject for one or more of the plurality of time windows.

21. (canceled)

22. (canceled)

23. A non-transitory computer readable storage medium seizure comprising instructions, which when executed by one or more processors, are configured to:
determine historical data associated with a subject experiencing epileptic events over a first time period, the historical data comprising non-EEG physiological data recorded over the first time period, and a time at which epileptic events occurred during the first time period;
extract from the non-EEG physiological data, one or more temporal models indicative of a subject specific cycle;
generate one or more temporal probabilistic models based on the respective one or more temporal models, the non-EEG physiological data, and the times at which epileptic event occurred, wherein each temporal probabilistic model is representative of a probability of future seizure activity in each of a plurality of time windows; and
provide the one or more temporal probabilistic models for determining an estimate of seizure probability in the subject for one or more of the plurality of time windows.
